(19) United States
(12) Patent Application Publication
(10) Pub. No.: US 2006/0030777 A1
(43) Pub. Date: Feb. 9, 2006

(54) T-statistic method for suppressing artifacts in blood vessel ultrasonic imaging

(76) Inventors: David H. Liang, Menlo Park, CA (US); Phillip Yang, Stanford, CA (US); Aditya Koolwal, Potomac, MD (US); Byong-Ho Park, San Jose, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 11/191,311

(22) Filed: Jul. 28, 2005

Related U.S. Application Data
(60) Provisional application No. 60/592,848, filed on Jul. 30, 2004.

(51) Int. Cl. A61B 8/00

(52) U.S. Cl. 600/437

(57) ABSTRACT

A technique for enhancing the image quality in intravascular ultrasound imaging increases contrast between blood and vessel wall processes image data using a point-wise t-statistic technique. Data from an ultrasound transducer is digitized and stored in a memory buffer [500]. For each point in the image, a t-statistic value is derived from signal amplitude values for the same point at a sequence of previous frames [502]. An image is then generated and displayed using the t-statistic values for the intensity of each point [504]. The improvement in contrast ratio as compared to averaging techniques is most significant at highly oblique angles when contrast ratio is particularly poor in the unprocessed signal.

### CROSS-REFERENCE TO RELATED APPLICATIONS

This application claims priority from U.S. provisional patent application No. 60/592,848 filed Jul. 30, 2004, which is incorporated herein by reference.

### FIELD OF THE INVENTION

This invention relates generally to methods and devices for ultrasound imaging. More specifically, it relates to signal processing techniques for enhancing the quality of images generated using very high frequency intravascular ultrasound.

### BACKGROUND OF THE INVENTION

Intravascular ultrasound is a medical imaging technique used in the study of blood vessels in vivo. A long and thin catheter is used to guide an ultrasound transducer through the interior of the blood vessel while computerized ultrasound equipment processes the ultrasound echoes and generates an image. Detailed information on the subject of intravascular ultrasonography is contained in U.S. Pat. No. 4,794,931 and U.S. Pat. No. 5,000,185, which are incorporated herein by reference.

Intravascular ultrasound involves imaging ultrasonic echoes at relatively short ranges. Consequently, it allows the use of very high frequency ultrasound (i.e., typically 20 to 40 MHz) which provides superb image resolution. At these high frequencies, however, the backscatter from blood increases, resulting in significant decreases in contrast ratio between the blood vessel wall and the lumen of the blood vessel. In the clinical use of intravascular ultrasound this decrease in contrast ratio is experienced frequently as the “loss of visualization” of the blood vessel wall, also referred to as “drop out”. FIG. 1A is an ultrasound image of a coronary blood vessel in vitro showing saline in the lumen 100 clearly contrasted with the vessel wall 102. The same blood vessel is shown in FIG. 1B with flowing blood in the lumen 104. The backscatter from the blood in lumen 104 dramatically reduces contrast between the lumen 104 and the vessel wall 106. It is thus desirable to remove or suppress the signal from the backscatter from blood to a level at which wall structures can be distinguished from blood.

One technique for increasing the contrast between the wall and lumen was proposed by Li, W., et al. in “Temporal averaging for quantification of lumen dimensions in intravascular ultrasound images.” Ultrasound Med Biol, 1994. 20(2): p. 117-22. This technique averages signals from successive image frames to smooth out the temporal variations of backscatter from flowing blood in the intraluminal ultrasound images, helping to increase contrast with the static signal from the vessel wall. However, such frame averaging results in only 20% reduction in the mean intensity of the backscatter, so it only partly reduces blood echoes from the image. FIG. 2A is an ultrasound image of the raw, unprocessed image of a blood vessel showing the lack of contrast between blood in the lumen 200 and the vessel wall 202. In comparison, FIG. 2B is a processed ultrasound image of a blood vessel, where the processing involves taking an average of multiple echoes. The contrast between the blood in lumen 204 and vessel wall 206 in this processed image is noticeably better than that of the raw image shown in FIG. 2A. The contrast, however, is less than perfect.

A similar approach also employing the temporal difference between the dynamic pattern of blood and static pattern of stationary vessel wall was proposed by Pasterkamp et al. in “Intravascular ultrasound image subtraction: a contrast enhancing technique to facilitate automatic three-dimensional visualization of the arterial lumen.” Ultrasound Med Biol, 1995. 21(7): p. 913-8. However, this technique subtracts the signals from the stationary vessel wall and retains the echo signals from the moving blood. It provides only the images of the blood lumen and is therefore of limited use.

A technique for blood noise reduction based on a beam tilting mechanism utilizing Doppler shift to separate the frequency signal from the blood and the vessel wall combined with the use of a lateral low pass filter of the blood signal was proposed by Gronningsaeter et al. in “Vessel wall detection and blood noise reduction in intravascular ultrasound imaging.” IEEE Trans Ultrason Ferroelect Freq Contr, 1994; 43:3:359-69. However, this technique is not applicable for low blood velocity and suffers from reduced lateral resolution without gray-scale. Subsequently, another method employing a spatial correlation technique based on probability density function between two adjacent frames to distinguish static and dynamic signals was also proposed by Gronningsaeter et al. in “Blood noise reduction in intravascular ultrasound imaging.” IEEE Trans Ultrason Ferroelect Freq Contr, 1995; 42:2:200-09. This approach, however, was limited by low spatial resolution, poor sensitivity to vessel wall motion, and the requirement of high frame rate.

A method combining temporal averaging with correlation techniques was proposed by Li, W., et al. in “Temporal correlation of blood scattering signals in vivo from radiofrequency intravascular ultrasound.” Ultrasound Med Biol, 1996. 22(5): p. 583-90. While the blood suppression was significantly improved, a significant trade-off requiring reduction of both frame-rate and angular resolution resulted.

Another technique for enhancing image quality is disclosed in U.S. Pat. No. 5,363,849, which is incorporated herein by reference. The method uses phase estimation and an analysis of multiple wavelengths. Unfortunately, this technique reduces the spatial resolution of the image. Moreover, the technique requires complex signal processing circuitry. Similar drawbacks also apply to techniques disclosed in U.S. Pat. No. 5,520,185 and U.S. Pat. No. 6,454,715.

In view of the above, there is a need for improved techniques for enhancing ultrasound images.

### SUMMARY OF THE INVENTION

In one aspect, the present invention provides a computationally efficient and effective technique for suppressing the time varying blood scatter signal and improving contrast in intravascular ultrasound imaging. By imaging the instantaneous t-statistic of repeated radiofrequency echoes, the lumen to blood vessel contrast is significantly improved as compared with averaging the radiofrequency of the repeated echoes. The technique is simple and fast to implement. Moreover, the improvement in contrast ratio can make feasible the use of forward-directed ultrasound beams. Because drop out is particularly severe at oblique angles between the blood vessel wall and the ultrasound beam, conventional intravascular ultrasound transducers direct pulses radially within the lumen rather than forward along the length of the vessel. With the significant improvement in contrast ratio at oblique angles provided by the technique of the present invention, however, forward-directed ultrasound beams become practical.

In one embodiment, a method for generating an enhanced ultrasound image from ultrasound echo amplitudes is provided. A temporal sequence of n image frames containing data samples representing the ultrasound echo amplitudes at image points in the frame are stored in a computer-readable memory and processed to produce an enhanced image. Portions of the enhanced image representing time-varying ultrasound echo amplitudes are suppressed to provide increased contrast between moving blood and the relatively still vessel wall. An image generated from the enhanced image is then displayed. The processing of the image frames includes calculating a point-wise t-statistic value for each image point. The t-statistic value for each image point may be calculated, for example, by computing a mean value of data samples for the image point in the n image frames, computing a standard deviation of data samples for the image point in the n image frames, and computing the ratio of the mean value to the standard deviation. This calculation is done point-wise, i.e., using sample data for individual points independent of data for other points in the image. Consequently, the calculation is simple and efficient. Moreover, the t-statistic method provides large contrast enhancement using only a few image frames, e.g., less than ten. Even with four or fewer frames significant enhancement is obtained, making the technique very fast to implement.

### BRIEF DESCRIPTION OF THE DRAWINGS

FIGS. 1A and 1B are ultrasound images of a coronary blood vessel containing saline and blood, respectively.

FIGS. 2A and 2B are ultrasound images a blood vessel before and after image processing by time averaging.

FIG. 3 is a schematic diagram of a generic ultrasound system which may be used to implement the techniques of the present invention.

FIGS. 4A and 4B are ultrasound images of a blood vessel processed using conventional time averaging and using the t-statistic technique of the present invention, respectively.

FIG. 5 is a flow chart of a technique of t-statistic image processing according to an embodiment of the present invention.

FIG. 6 is a graph of the wall-to-blood contrast ratio vs. number of image frames used in a t-statistic technique of the present invention.

FIGS. 7A and 7B are ultrasound images processed using just four frames using time averaging and the t-statistic technique, respectively.

FIG. 8 is a graph of the mean signal intensity reflected from a blood vessel wall vs. angle of incidence.

FIGS. 9A-C are graphs of signal amplitude vs. echo delay at 30° angle of incidence for raw unprocessed data, time-averaged data, and t-statistic processed data, respectively.

FIG. 10 is a graph of vessel wall-to-blood contrast signal (dB) vs. angle of incidence for raw, time-averaged, and t-statistic data.

### DETAILED DESCRIPTION

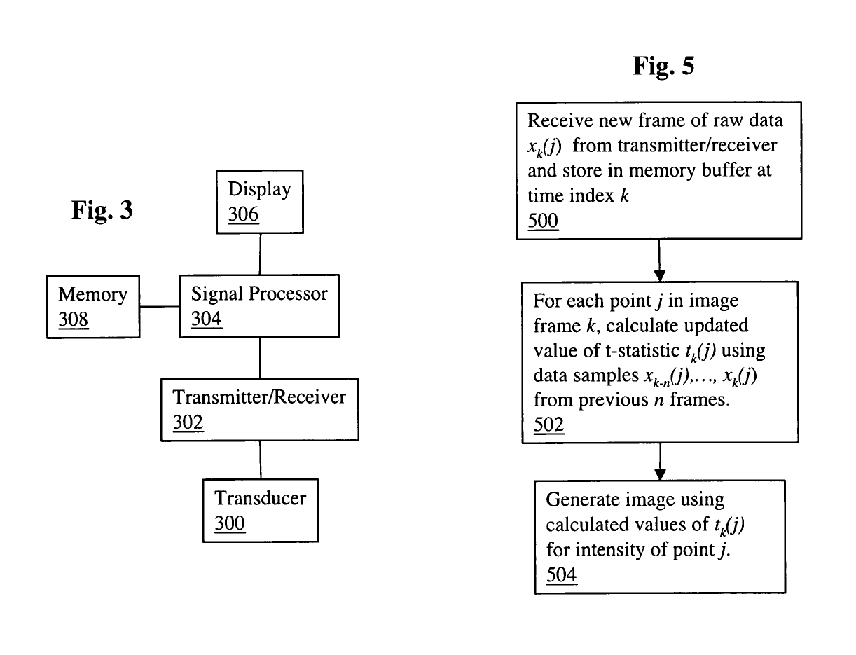

Embodiments of the present invention may be implemented using various types of intravascular ultrasound systems, suitably modified to process signals as will be described in more detail later. A schematic diagram of a generic ultrasound system is shown in FIG. 3. An ultrasound transducer 300 is connected to a transmitter/receiver 302. A signal processor 304 connected to transmitter/receiver 302 processes the signals, stores them in connected memory 308, and produces a digital image for viewing on connected display 306. Transducer 300 is conventionally attached to the end of a catheter which may be inserted into a blood vessel. Various types of transducer 300 may be used, including sideways-directed, forward-directed, and a combination of both. Signal processor 304 may be a programmable digital signal processor (DSP) or other processor built into an ultrasound imaging device, or it may be software running on a conventional desktop computer. Ultrasound systems may manifest the generic components described above in various configurations, as is well known in the art.

In operation, transmitter/receiver 302 may generate, for example, a 30 MHz electrical pulse that drives transducer 300 to generate corresponding ultrasonic waves. Echoes of the ultrasonic waves reflected back to the transducer 300 are converted to electrical signals representing the amplitude of the reflected pulses. These signals are received by transmitter/receiver 302 where they are preamplified, filtered, digitized, and passed on to signal processor 304 in real time.

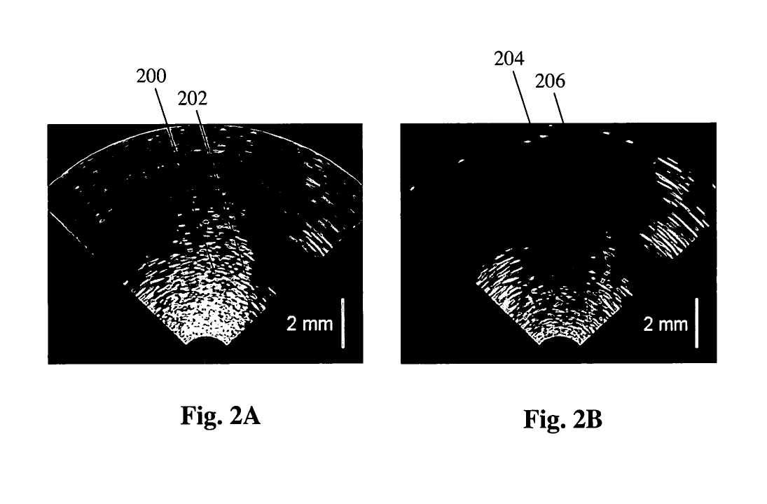

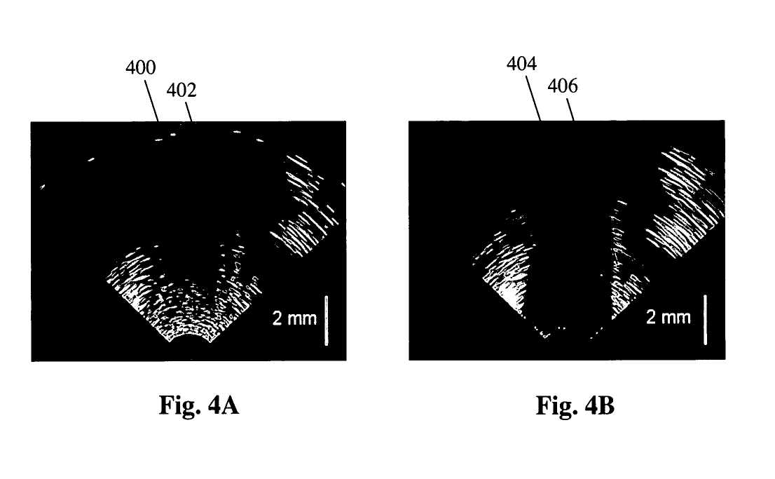

The raw amplitude data arriving at signal processor 304 may be processed in various ways to improve the visualizability of image features. FIG. 2A shows an example of raw image data without any such processing. FIG. 2B shows an example of an image processed by time-averaging, showing slightly improved contrast between the blood and the vessel wall. The present invention provides a t-statistic technique for processing the raw image data that provides significantly better contrast than time averaging, as illustrated by comparison of FIGS. 4A and 4B. The ultrasound image in FIG. 4A is processed using conventional time averaging. In comparison, FIG. 4B is an image processed using the t-statistic technique of the present invention. As clearly illustrated by the figures, the contrast between lumen 404 and wall 406 in the image processed with the t-statistic technique is far superior to the contrast between lumen 400 and wall 402 in the image processed with averaging.

In brief, this t-statistic technique calculates, for each point in the image, a t-statistic value from a temporal sequence of raw amplitude values for that point. The t-statistic is then used to form the displayed image, either directly or in combination with additional processing. This approach significantly reduces the blood signal beyond that achievable with simple averaging and restores adequate lumen to blood vessel wall contrast to angles of incidence as great as 60 degrees from perpendicular.

A specific t-statistic technique according to one embodiment of the invention will now be described in more detail. Each point in the raw image data arriving at the signal processor corresponds to a particular echo delay and scan angle. If the amplitude data at a particular point is representative of an echo signal from the blood, then the mean of the data at that point over time will be zero due to the random phase of the returned echo from the moving blood. If, on the other hand, the amplitude data at the point is representative of an echo from the vessel wall, then the mean of the data at that point over time will have a non-zero mean, due to the non-random phase of reflections from the stationary vessel wall. The task of discriminating blood flowing blood from stationary wall is then equivalent to discriminating zero mean from non-zero mean. The maximum likelihood test statistic for performing this task is the t-statistic. The t-statistic value tₖ(j) for a particular image point identified with index j at a particular time indexed by k may be described mathematically by the following equation:

$${t}_{k} ( j ) = \frac{{Mean}_{k} ( j )}{{SD}_{k} ( j )} = \frac{\frac{1}{n} {\sum }_{i = k - n , k} {x}_{i} ( j )}{\sqrt{\frac{1}{n - 1} {\sum }_{i = k - n , k} {( {x}_{i} ( j ) - \frac{1}{n} {\sum }_{i = k - n , k} {x}_{i} ( j ) )}^{2}}} \qquad ( eq . 1 )$$

where xᵢ(j) is the amplitude value at image point j at time index i, and n is the number of time samples (i.e., echoes) used.

A signal processor or computer 304 of an ultrasound imaging system (FIG. 3) may implement the technique using the steps shown in the flow chart of FIG. 5. In step 500 the processor 304 receives a new frame of raw image data from transmitter/receiver 302 and stores it in memory 308 buffer with a time index k. This raw data is represented as {xₖ(j): j=1, . . . ,N} where N is the number of points in each frame. At step 502 the technique uses equation to calculate, for each point j in image frame k, an updated value of a t-statistic value tₖ(j) using data samples xₖ₋ₙ(j), . . . , xₖ(j) from the previous n frames of data. An image for display is then generated in step 504 using the calculated values of tₖ(j) for intensity of image point j. A mapping function from tₖ(j) to image intensity may also be used prior to display to enhance perceptibility of differences in the some regions in the range of tₖ(j) values to enhance visualization of desired anatomic features.

Note that with certain ultrasonic scanner designs (e.g., mechanically scanned intravascular ultrasound systems), individual echoes can be obtained much more rapidly than complete frames due to the short propagation and the relatively slow sweep of the transducer beam. Consequently, multiple image points may be acquired in a given direction before the beam is moved to a new direction. More generally, the order of acquisition of image points may differ between various ultrasound systems.

Note that the t-statistic calculation step 502 may efficiently calculate the t-statistic value by first calculating the value of Meanₖ(j) and then using this value in the calculation of SDₖ(j). In addition, the value of Meanₖ(j) can be efficiently updated for frame k without recalculating the n-term sum using the relationship

$${Mean}_{k + 1} ( j ) = {Mean}_{k} ( j ) + \frac{1}{n} ( {x}_{k + 1} ( j ) - {x}_{k - n} ( j ) ) \qquad ( eq . 2 )$$

Those skilled in the art will appreciate that this is just one particular example of how the t-statistic value tₖ(j) may be calculated, and that many other equivalent ways of calculating the t-statistic may be used. It will also be appreciated that the t-statistic image values tₖ(j) may be further processed prior to displaying the image using any of various well-known image processing techniques known in the art of ultrasound imaging. Such techniques may also be used to pre-process the raw data Xₖ(j) prior to calculating the t-statistic.

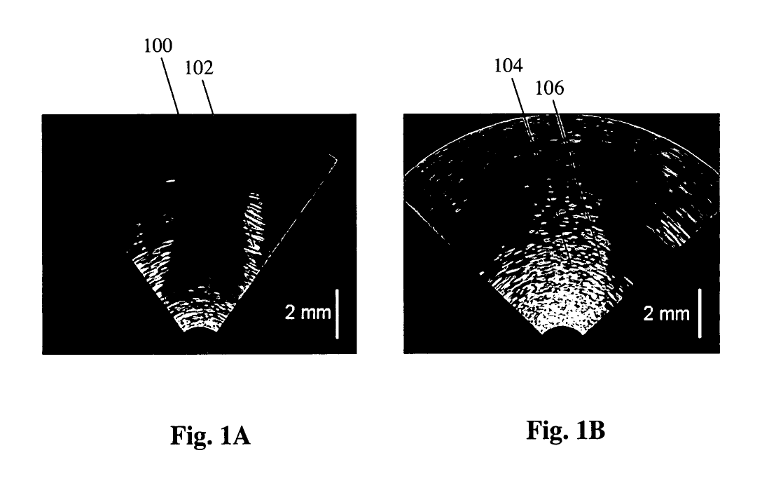

FIGS. 4A and 4B are intravascular ultrasound images illustrating the improvement of the image quality generated from the t-statistic (FIG. 4B) over the quality of the averaged image (FIG. 4A). The image generated from the t-statistic approaches the quality of the image generated in saline (FIG. 1A).

An important property of this statistical technique is that as n increases, the value of the t-statistic tₖ(j) rises or falls rapidly, depending on whether the point j has a non-zero mean or zero mean. Imaging using the t-statistic with suitable n thus provides suppression of the time varying portions of the image and high contrast between blood and vessel wall. For stationary signals the denominator of the t-statistic will be primarily generated by the random noise is the ultrasound system. This value should be relatively constant across the image, so the stationary portions of the image should suffer relatively little distortion.

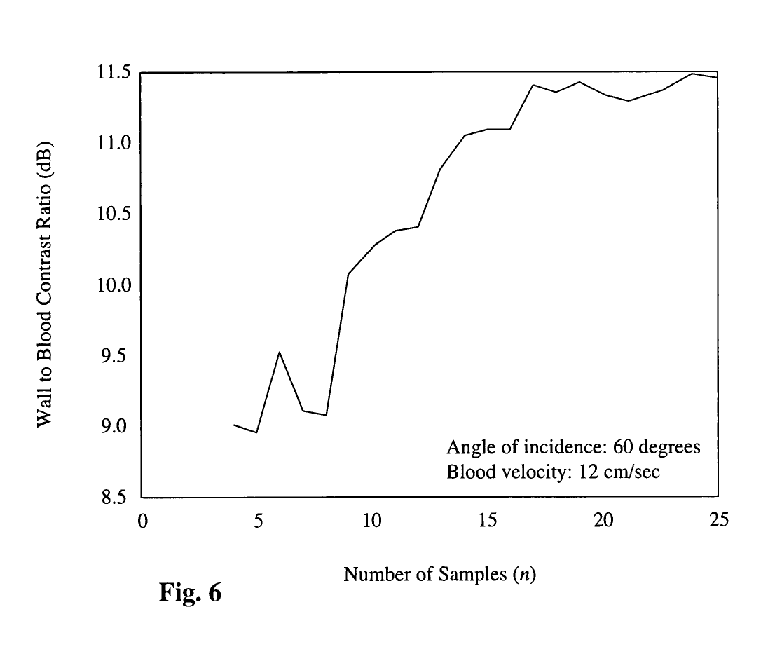

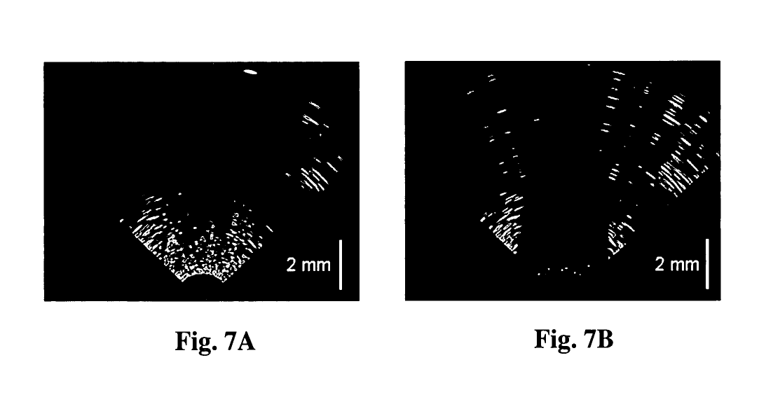

One of the principal advantages of t-statistic imaging over averaging is the rapidity with which blood signal is suppressed, allowing fewer echoes to be used per image. FIG. 6 is a graph of the wall-to-blood contrast ratio vs. number of samples (n) which shows the improvement in contrast between blood vessel wall and blood with use of increasing numbers of echoes in calculating the t-statistic. Significant improvement in contrast are seen with as few as four echoes (i.e., n=4), as illustrated by comparing images in FIGS. 7A and 7B. In FIG. 7A the image is averaged over four frames (i.e., echoes) while in FIG. 7B the image is processed using the t-statistic over four frames. The advantage of t-statistic imaging over averaging is thus even more apparent with small echo number. With just a few time samples, the t-statistic method provides significant enhancement of image contrast with very few calculations. In addition, because the t-statistic method involves a point-wise computation, it is computationally efficient and does not reduce image resolution.

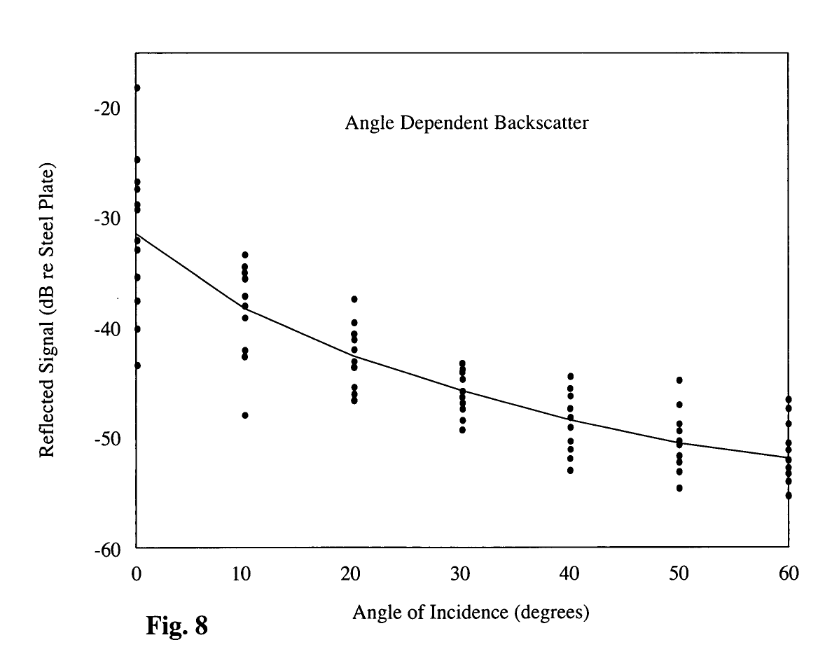

Another important advantage of the t-statistic method is seen in its effectiveness to enhance image contrast at high angles of incidence, which are characteristic of forward-viewing intravascular ultrasound systems (e.g., U.S. Pat. No. 5,373,849 and U.S. Pat. No. 5,606,975, which are incorporated herein by reference). In forward-viewing intravascular ultrasound the angle of incidence of the ultrasound on the blood vessel wall deviates from perpendicular to a much greater degree than in conventional side-viewing intravascular ultrasound. Consequently, “drop out” is a much more severe problem in forward-viewing scanning than for standard radially oriented scanning. For example, in FIG. 8 the mean signal intensity reflected from a blood vessel wall is graphed as a function of the angle of incidence from 0° to 60° in normal saline. The reflected signal strength demonstrates a rapid decline as the angle of incidence of the ultrasound becomes less perpendicular to the blood vessel wall. The decline is approximately 3.2 dB/degree. Due to this reduced signal strength from the vessel wall at large angles of incidence, it is important for the feasibility of forward-viewing ultrasound that effective techniques be developed for significantly reducing backscatter signals from blood at high angles of incidence.

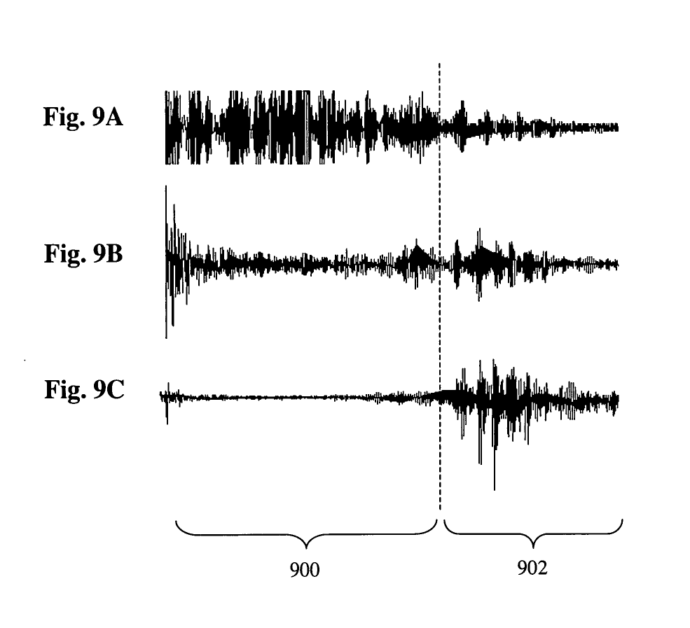

The RF data obtained at 30° angle of incidence is shown in FIGS. 9A-C, which are graphs of signal amplitude vs. echo delay (i.e., distance from the transducer). The raw signal (FIG. 9A) shows no discrimination in the signal amplitude between blood in the lumen 900 and the vessel wall 902. The signal from blood is larger amplitude than the signal from the vessel wall. The averaged RF signal (FIG. 9B) over several echoes enables identification of the vessel wall signal, but the contrast is not high. The t-statistic (FIG. 9C) shows significant additional improvement in contrast between the vessel wall 902 and blood in the lumen 900.

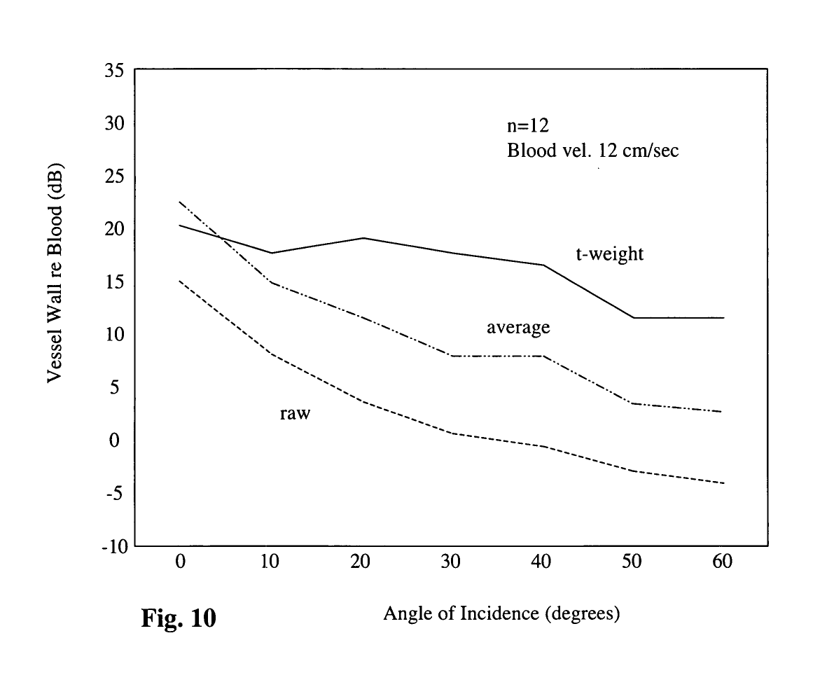

When the vessel wall to blood contrast signal (dB) is plotted as a function of the angle of incidence, as shown in FIG. 10, the enhancement of the signal contrast demonstrated by the t-weighted data over the raw and averaged data becomes particularly apparent as the angle of incidence becomes more oblique. From 20° angle of incidence, there is approximately 15 dB improvement of contrast signal when comparing the t-weighted signal to the raw data and approximately 8 dB improvement from the t-weighted to the averaged data. Thus, the present invention is particularly useful in forward-viewing systems where angles of incidence are high. Forward viewing capability provides several advantages. First, it allows imaging of a lesion in front of the catheter as it moves further down the vessel. Second, it provides improved imaging of the course of a totally occluded blood vessel providing guidance on the length, direction, and extent of calcification of the lesion. Finally, this modality enables real-time imaging of intravascular intervention and helps minimize unnecessary injury to the vascular tissue. Interventional devices operating in forward direction such as laser, rotational atherectomy, and rotablator may benefit.

In summary, the optimal t-weighted signal processing technique described above enhances the contrast between blood and vessel wall in intravascular ultrasound. The use of t-statistics suppresses the blood signal much more rapidly that other known techniques, such as averaging, and provides significant improvement in image processing applicable to forward viewing modality. The calculation is relatively simple allowing implementation in real time using simple hardware.

1. A method for generating an enhanced ultrasound image from ultrasound echo amplitudes, the method comprising:
storing in a computer-readable memory a temporal sequence of n image frames comprising data samples representing the ultrasound echo amplitudes at image points in the frame;
processing the temporal sequence of image frames to produce an enhanced image wherein portions of the enhanced image representing time-varying ultrasound echo amplitudes are suppressed; and
displaying an image generated from the enhanced image;
wherein the processing includes calculating a point-wise t-statistic value for each image point.

2. The method of claim 1 wherein calculating the t-statistic value for each image point comprises computing a mean value of data samples for the image point in the n image frames, computing a standard deviation of data samples for the image point in the n image frames, and computing the ratio of the mean value to the standard deviation.

3. The method of claim 1 wherein calculating the t-statistic value for each image point comprises calculating a value of tₖ(j) defined by
$${t}_{k} ( j ) = \frac{\frac{1}{n} {\sum }_{i = k - n , k} {x}_{i} ( j )}{\sqrt{\frac{1}{n - 1} {\sum }_{i = k - n , k} {( {x}_{i} ( j ) - \frac{1}{n} {\sum }_{i = k - n , k} {x}_{i} ( j ) )}^{2}}} ,$$
where j is an index for the image point, i is an index for the n image frames, k is an index for a most recent image frame in the n image frames, and xᵢ(j) is a data sample value representing an ultrasound echo amplitude at image point j in frame i.

4. The method of claim 1 wherein the value of n is no more than four.

5. The method of claim 1 wherein the value of n is no more than ten.

6. A ultrasound imaging device comprising an ultrasound transducer, a transmitter/receiver connected to the transducer, a signal processor connected to the transmitter/receiver, a memory connected to the signal processor, and a display connected to the signal processor, wherein the signal processor comprises instructions for:
storing in the memory a temporal sequence of n image frames comprising data samples representing the ultrasound echo amplitudes at image points in the frame;
processing the temporal sequence of image frames to produce an enhanced image wherein portions of the enhanced image representing time-varying ultrasound echo amplitudes are suppressed; and
displaying an image generated from the enhanced image;
wherein the processing comprises calculating a point-wise t-statistic value for each image point.

7. The device of claim 6 wherein calculating the t-statistic value for each image point comprises computing a mean value of data samples for the image point in the n image frames, computing a standard deviation of data samples for the image point in the n image frames, and computing the ratio of the mean value to the standard deviation.

8. The device of claim 6 wherein calculating the t-statistic value for each image point comprises calculating a value of tₖ(j) defined by
$${t}_{k} ( j ) = \frac{\frac{1}{n} {\sum }_{i = k - n , k} {x}_{i} ( j )}{\sqrt{\frac{1}{n - 1} {\sum }_{i = k - n , k} {( {x}_{i} ( j ) - \frac{1}{n} {\sum }_{i = k - n , k} {x}_{i} ( j ) )}^{2}}} ,$$
where j is an index for the image point, i is an index for the n image frames, k is an index for a most recent image frame in the n image frames, and xᵢ(j) is a data sample value representing an ultrasound echo amplitude at image point j in frame i.

9. The device of claim 6 wherein the value of n is no more than four.

10. The device of claim 6 wherein the value of n is no more than ten.
